(19) United States
(12) Patent Application Publication
(10) Pub. No.: US 2016/0185566 A1
(43) Pub. Date: Jun. 30, 2016

(54) Roll Material Dispenser and Method

(71) Applicant: Medline Industries, Inc., Mundelein, IL (US)

(72) Inventor: Tambra Martin, Trevor, WI (US)

(21) Appl. No.: 14/586,532

(22) Filed: Dec. 30, 2014

(51) Int. Cl. B65H 75/16; A61F 5/01; B65H 16/00

(52) U.S. Cl. CPC B65H 75/16; B65H 16/005; A61F 5/0102; B65H 2701/50; B65H 2701/194

(57) ABSTRACT

Disclosed are a roll material dispenser and method of dispensing a roll material. The roll material dispenser comprises a container having a generally cylindrical body and cover and first and second slits disposed in the wall surface, and a roll of roll material disposed within the interior of the container. Roll material may be obtained by dispensing the roll material through a first slit, removing a portion of the material so dispensed, and tucking a lead end of the roll material into the second slit.

### TECHNICAL FIELD

The present disclosure generally relates in some aspects to a roll material dispenser and in other aspects to methods for dispensing a roll of roll material from a container. The roll material may for example be elastic therapeutic tape.

### BACKGROUND

Elastic therapeutic tape generally comprises a strip of an elastic material, typically composed in part of cotton with an adhesive backing Such tape is intended to be used to treat athletic injuries and other physical disorders. Typical elastic therapeutic tape is able to be stretched to a substantial percentage greater than its original length, such that, if the tape is supplied to a patient upon stretching, the tape will recoil and therefore create a pulling force on the patient's skin. Additionally, the elastic property allows a larger range of motion compared to non-elastic typical athletic tape. Elastic therapeutic tape typically is provided with a pressure-sensitive adhesive that is activated by pressing or rubbing after application.

Elastic therapeutic tape is typically provided in the form of pre-cut strips. It is desirable to provide a container that protects the tape from damage during storage prior to use. In certain prior dispensing systems, a complete roll of tape must be removed from the container to thereby allow removal of a single pre-cut strip of the tape. This can cause soiling of and damage to subsequent strips on the roll of tape once the user has removed and applied the desired number and strips.

### SUMMARY

In certain aspects, the invention provides a roll material dispenser that is useful in conjunction with dispensing elastic therapeutic tape and other roll materials. The dispenser generally comprises a container and a roll of a roll material disposed within the interior of the container. The container includes a generally cylindrical body that defines a wall surface and a substantially hollow interior, and having a cover connected to the cylindrical body. Preferably, the cover is detachable from and re-connectable to the cylindrical body. The body includes a first slit disposed in the wall surface and having a first slit height and a second slit disposed in the wall surface and having a second slit height, the second slit being radially spaced from the first slit. The roll material has a lateral width, whereby the lateral width of the roll material and the heights of the first and second slits are suitable dimensioned to permit dispensing of the roll material from the container through the first slit and to permit a lead portion of the roll material to be tucked into the second slit to thereby retain the lead portion. Usually, the axial slit height of each slit will be greater than the lateral width of the roll material. The roll material may be elastic therapeutic tape or any other suitable roll material.

A method of preparing a container of roll material is also provided in certain other aspects. Generally, this method comprises providing a container, providing a roll of roll material, inserting the roll of roll material into the container, and covering an opening of the container with a cover. The container and roll material generally are as described hereinabove.

Further, in certain other aspects, the invention provides a method that includes providing a roll material dispenser as described above, dispensing roll material through the first slit, removing a portion of the dispensed roll material to leave a lead portion extending from the first slit, the lead portion having a lead end, and tucking the lead end into the second slit to thereby retain the lead portion.

Further aspects of the disclosure are described herein and illustrated in the appended drawings.

### BRIEF DESCRIPTION OF THE DRAWINGS

FIG. 1 is a perspective view of an exemplary roll material dispensing system, including a roll of elastic therapeutic tape.

FIG. 2 is a perspective view of the roll material tape dispensing system shown in FIG. 1 shown with a first end cap removed and placed to the side.

FIG. 3 is a perspective view of the canister of the roll material dispensing system shown in FIG. 1, shown with the roll of elastic therapeutic tape removed.

FIG. 4 is an inside plan view of the end cap of the roll material dispensing system shown in FIGS. 1 and 2.

FIG. 5 is a side elevation of the end cap of the roll material dispensing system shown in FIGS. 1 and 2.

FIG. 6 is a perspective view of the canister of the roll material dispensing system shown in FIG. 1, shown with both end caps removed.

FIG. 7 is an exploded view of the roll material dispensing system shown in FIG. 1.

FIG. 8 is a perspective view of the roll of elastic therapeutic tape.

FIG. 9 is a perspective view of the roll of elastic therapeutic tape, including protective wrapping for shipment to the consumer.

FIG. 10 is a plan view of a partially unrolled section of the roll of elastic therapeutic tape from the roll shown in FIGS. 8 and 9, showing plural precut strips of tape.

FIG. 11 is a plan view of a partially unrolled section of an alternative embodiment of the elastic therapeutic tape roll.

FIG. 12 is a perspective view illustrating an end of the roll of elastic therapeutic tape shown in FIG. 10 emerging from a first slit of the canister shown in FIG. 1.

FIG. 13 is a perspective view illustrating the end of the roll emerging from the first slit of the canister, further along in one optional process of dispensing the roll material.

FIG. 14 is a perspective view illustrating a lead end of the roll material tucked into the second slit of the canister.

FIG. 15 is a view similar to FIG. 14 except showing the alternative tape design illustrated in FIG. 11.

FIG. 16 is a perspective view of a pre-cut strip of elastic therapeutic tape shown being peeled away from a backing material.

FIG. 17 is a perspective view illustrating an alternative canister design having reduced slit heights.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention.

### DESCRIPTION

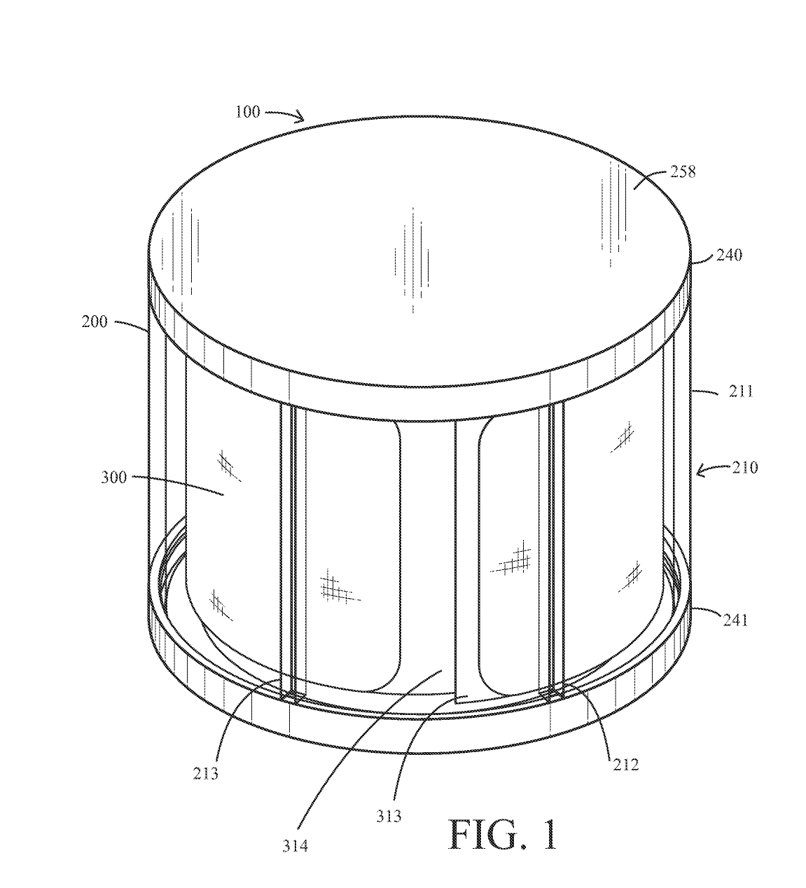

Referring now to the drawings, and in particular to FIG. 1, a roll material dispenser 100 includes a canister forming a container 200 and a roll of roll material 300. The container 200 includes a generally cylindrical body 210 defining a wall surface 211. Preferably, the cylindrical body 210 is translucent, allowing a user to view the contents contained within the substantially hollow interior of the cylindrical body 210 and permitting great variation in the placement of graphic and instruction labels. The cylindrical body 210 may be formed of any rigid material, and preferably is formed from a resinous base material such as polyethylene terephthalate (PET or PETE), high density polyethylene (HDPE), polyvinyl chloride (PVC), low density polyethylene (LDPE), polypropylene (PP), or polystyrene (PS).

The body may have any suitable dimensions and in some embodiments the radius of the cylindrical body 210 may be between 70 and 90 millimeters. Disposed in the wall surface 211 of the cylindrical body 210 are a first slit 212 and a second slit 213. The first and second slits 212, 213 define openings in the wall surface 211 of the cylindrical body 210 such that a lead portion 313 of roll material 310 having a lead end 314 may be passed to and from the interior of the container 200 through the slits 212, 213. When the wall surface is generally cylindrical, the slits preferably track a radial planar intersection line with the wall surface, as shown. The wall surface need not be cylindrical and the slits may take any other appropriate form. Preferably, and as shown in FIG. 1, the slits take an axially linear oriented form roughly commensurate with generating cylinder lines of the wall surface. The slits 212, 213 are generally parallel to one another and are radially spaced or offset from one another in any suitable degree of separation. As shown, the first slit 212 is radially spaced from the second slit 213 by approximately 60°. The slits may take other forms so long as they are sufficiently radially offset from one another to permit the dispensing and tucking actions described in more detail below.

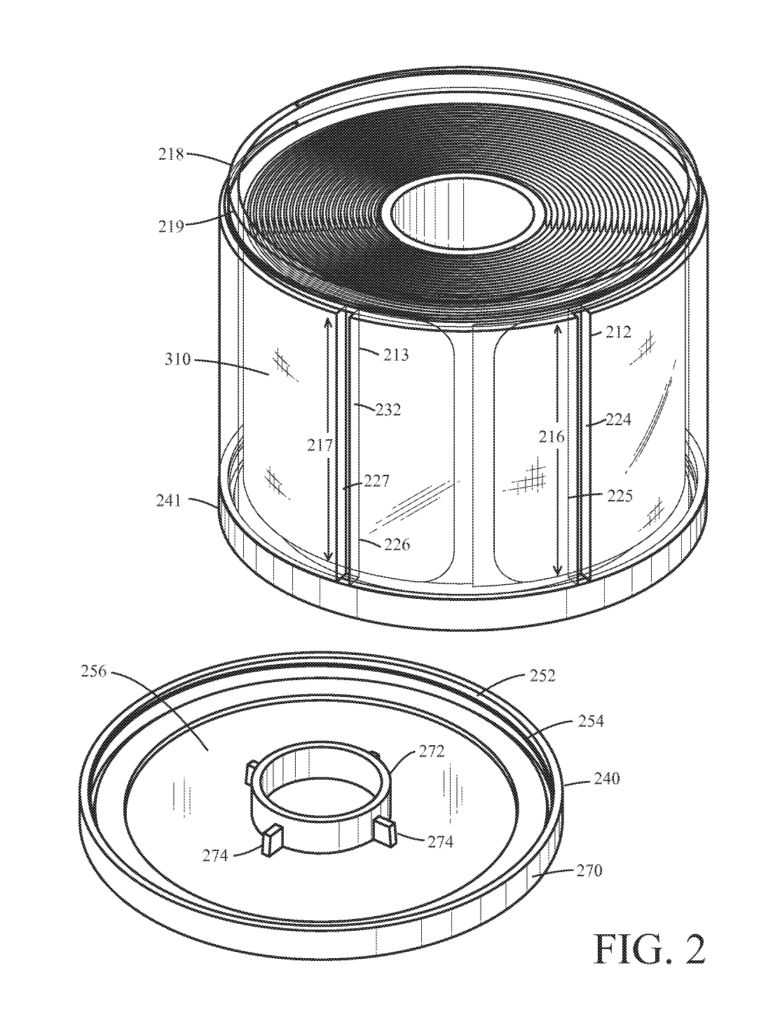

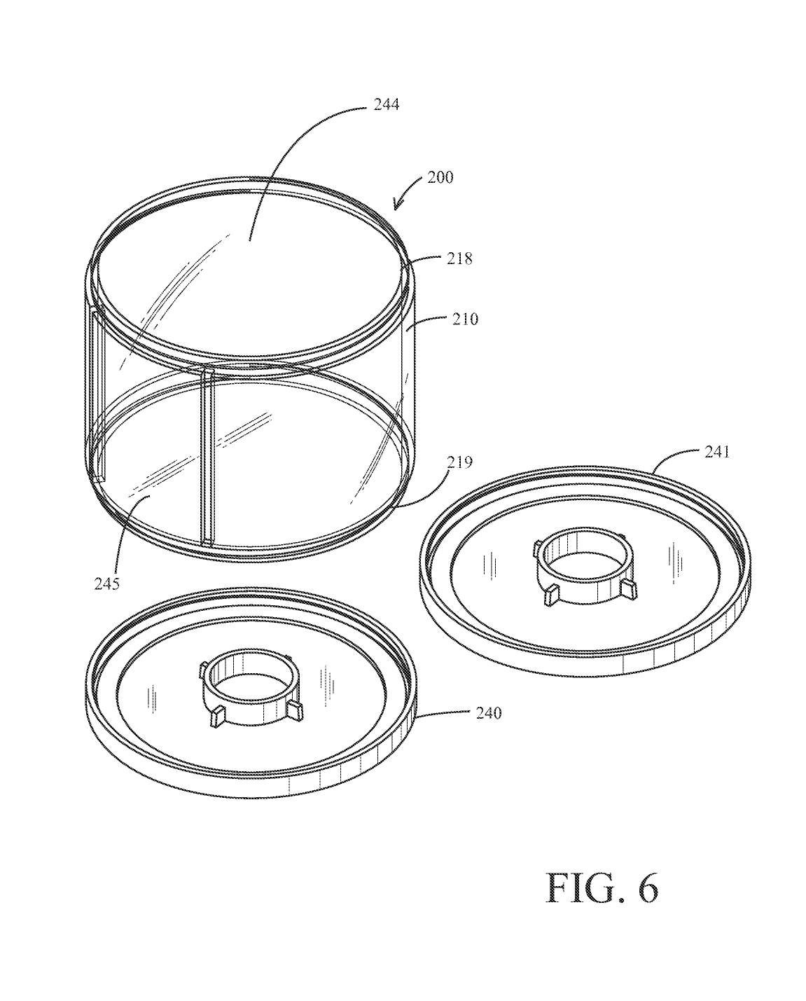

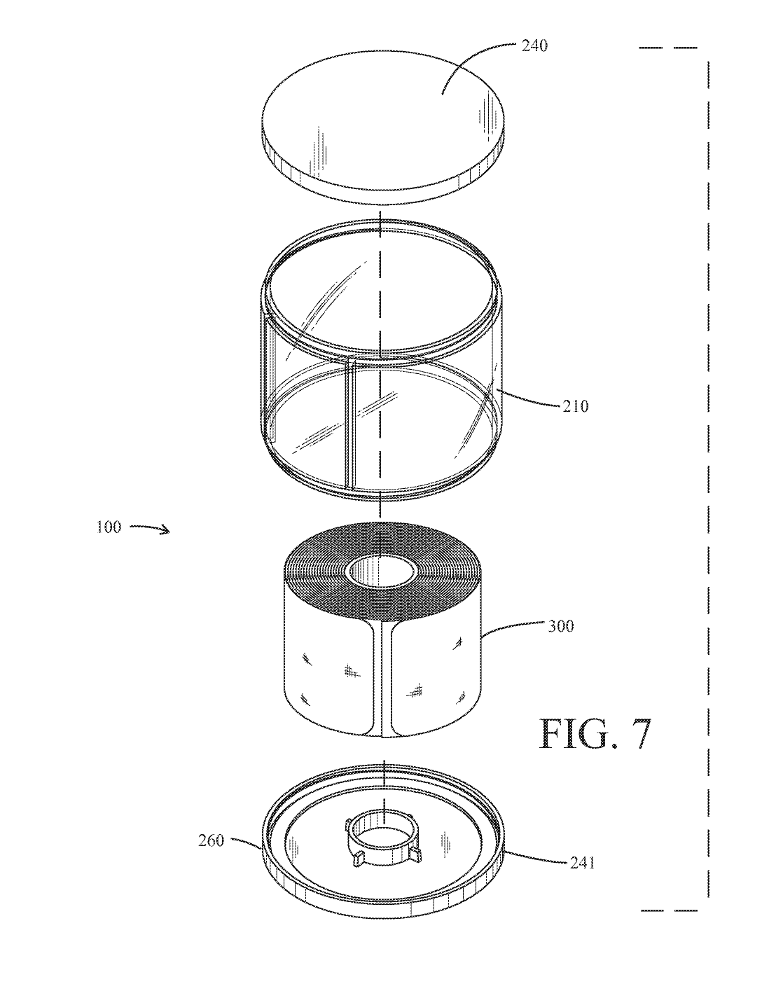

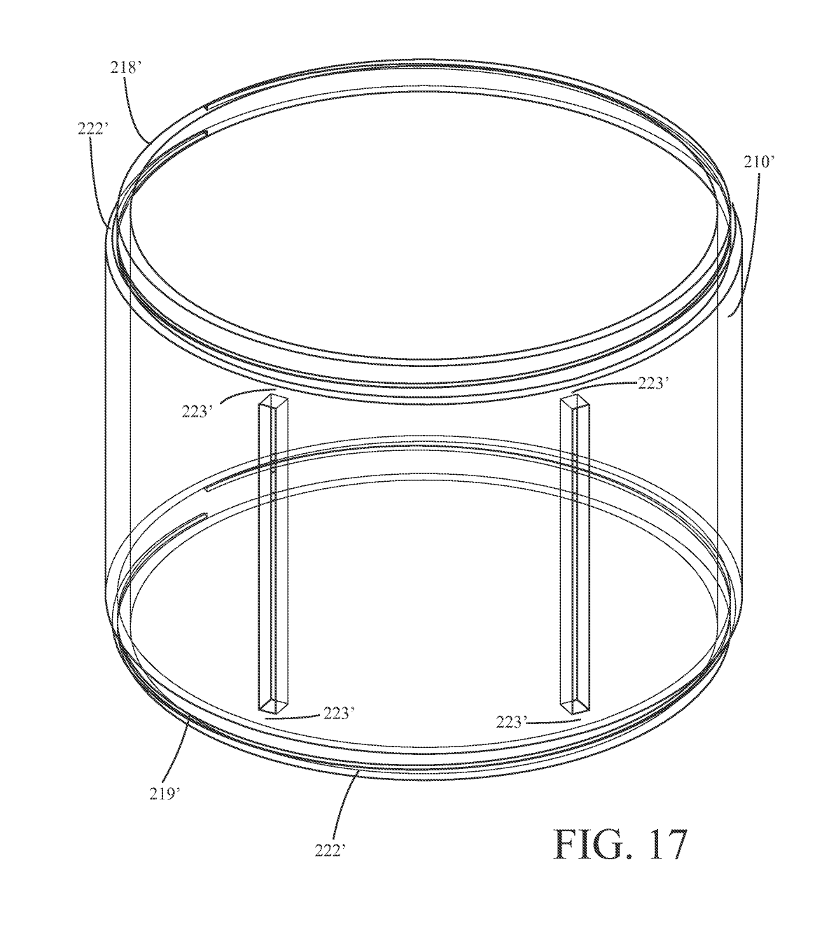

Each slit 212, 213 is dimensioned as a slender linear opening having a slit height (216, 217 in FIG. 2), by which is contemplated the full axial extent of the slit. As illustrated, each slit 212, 213 has a slit height between 50 and 60 millimeters. Preferably, as depicted in FIG. 6, each slit 212, 213 has a slit height that is between 80% and 90% of the total axial height of the container 200 and that extends fully between securement regions 218, 219 of the top and bottom of the cylindrical body 210. Alternatively, as depicted in FIG. 17, the slits may not extend fully between the securement regions 218′, 219′ of the top and bottom of the cylindrical body 210′. In this approach, the slits are separated from top and the bottom ledge 222′ by intermediate areas 223′.

The radial width of each slit may have any suitable dimension and for example may be between 2 and 3 millimeters. As shown, and with reference to FIG. 2, each slit has side walls 224, 225, 226, 227 that extend generally radially, although oblique configurations are possible.

Preferably, the configuration of the first slit 212 (e.g., size, shape, angular orientation, wall orientation, and slit height) are the same as those of the second slit 213. In some approaches, however, the properties of the first slit differ from those of the second slit or from the illustrated embodiment. For example, the slits may have different dimensions, or one or both of the slits may take the track of an oblique plane intersection curve on the wall surface.

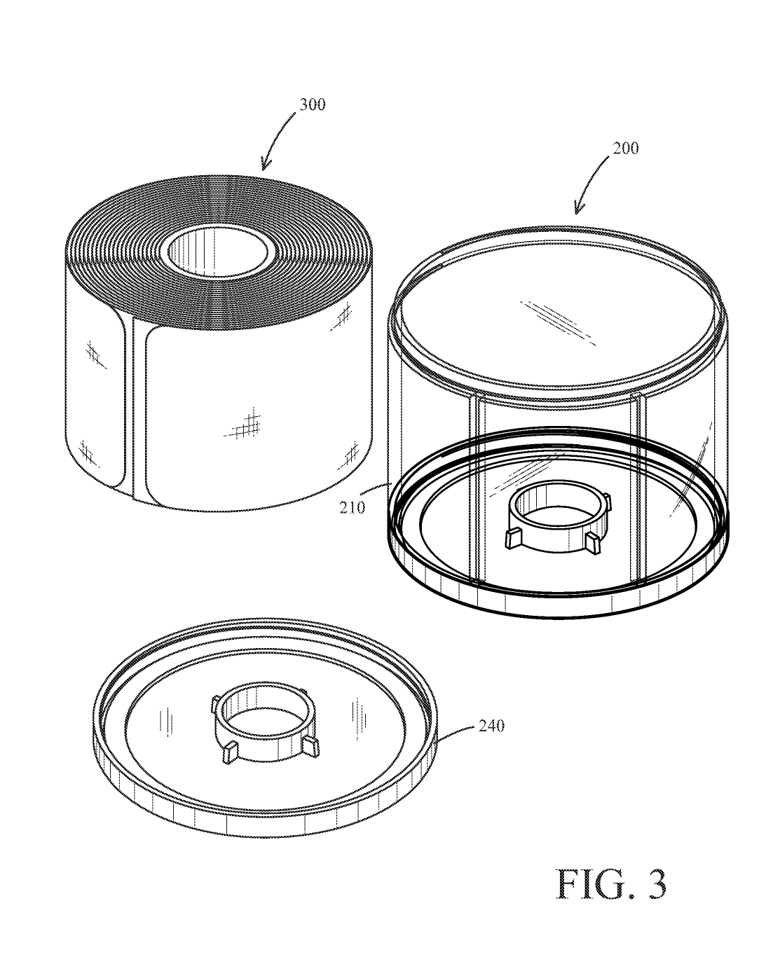

The container 200 in the illustrated embodiment includes a first cover 240 and a second cover 241 opposing the first cover 240. As shown in FIGS. 2 and 3, at least one of the first cover 240 and second cover 21 is removable from the cylindrical body 210, this to allow the user to access the contents of the container 200. Preferably, the container 200 includes two removable covers 240, 241. The two removable covers 240, 241 may be identical in appearance, or may differ in including the various cover features described herein. The first and second covers 240, 241 cover the first and second circular openings 244, 245 (shown in FIG. 6) in the cylindrical body 210. In this way, the first cover 240 and second cover 241 contain the contents of the container 200 within the container 200. In some embodiments, the container is provided with a single opening and cover. For example, instead of the second opening 245, the container may be provided with a closed wall surface. The cover may be composed of any suitable material and may be composed of the same material as the body.

The covers 240, 241 may be detachable from and reconnectable to the cylindrical body 210 using any suitable type of securement. Preferably, and with particular reference to FIG. 2, the cylindrical body 210 includes a securement region 218 having at least one thread 219, and the cover 240 likewise includes a securement region 252 having at least one thread 254 capable of threadably mating with the thread 219 of the cylindrical body 210. In another approach, the cover 240 is secured to the cylindrical body 210 using a “snap-fit” securement. In this approach, the securement region of the cover includes a protrusion (such as an inward projecting rib or ridge), and the securement region of the cylindrical body includes a complementary protrusion (such as an outward projecting rib or ridge). This approach advantageously allows a user to hear an audible “snap” when the cover is secured to the cylindrical body, thereby indicating the container has properly been sealed.

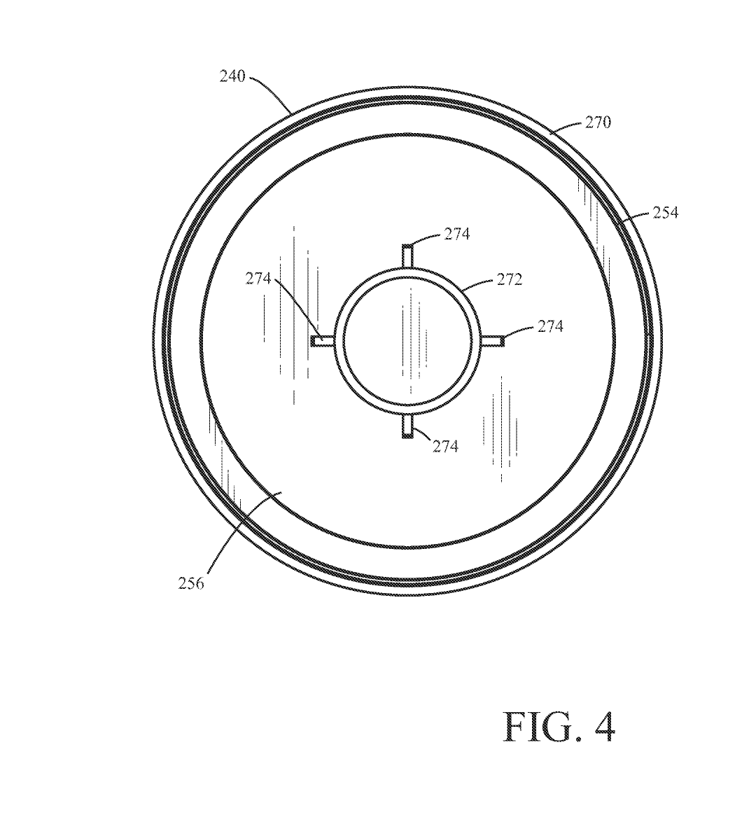

With reference to FIGS. 1, 2 and 4, the cover 241 includes an internal base surface 256 (shown in FIG. 4) and an external surface 258 (shown in FIG. 1). The cover 241 also includes a lip 270 generally extends orthogonally from to the internal base surface 256 in an internal direction.

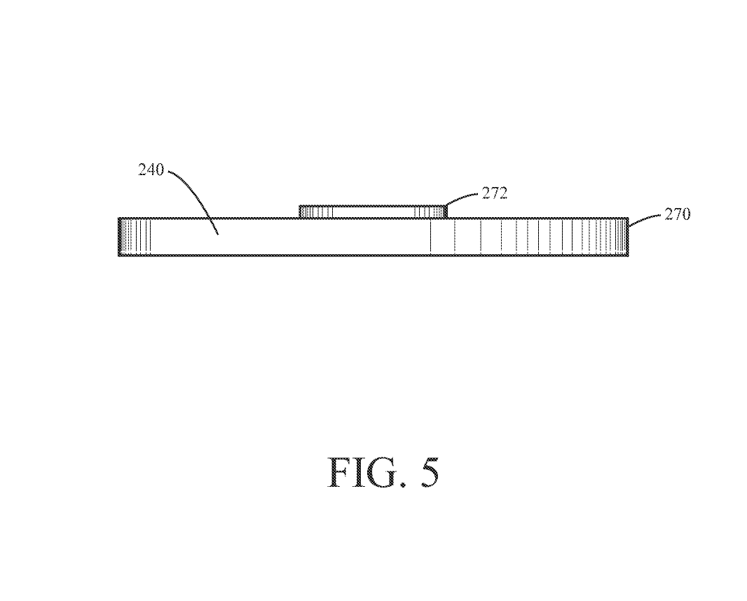

Preferably, as seen in FIGS. 2, 4 and 5, at least one of the first cover 240 and the second cover 241 includes a protrusion 272 extending away from the cover in an internal direction. The protrusion may be disposed on a closed end of the cylindrical body when the cylinder is provided with a single cover. When the container 200 is closed, the protrusion 272 extends into the interior of the cylindrical body 210. As shown in FIG. 5, the protrusion 272 extends from the internal base surface 256 to a greater extent internally away from the internal base surface 256 than the lip 270. The protrusion 272 is sized and positioned to inhibit radial motion of the roll of roll material 300, as discussed in greater detail below. For example, the protrusion 272 may be in the form of a hollow or solid cylindrical boss. The protrusion may also serve to provide additional rigidity to the cover 240.

With further reference to FIGS. 2 and 6, at least one of the first cover 240 and the second cover 241 may include at least one axial retaining member 274 extending inwardly away from the internal base surface and preferably proximal the protrusion 272. The axial retaining members 274 are sized and positioned to inhibit axial motion of the roll of roll material 300 at least when the container 200 is closed, as discussed in more detail below.

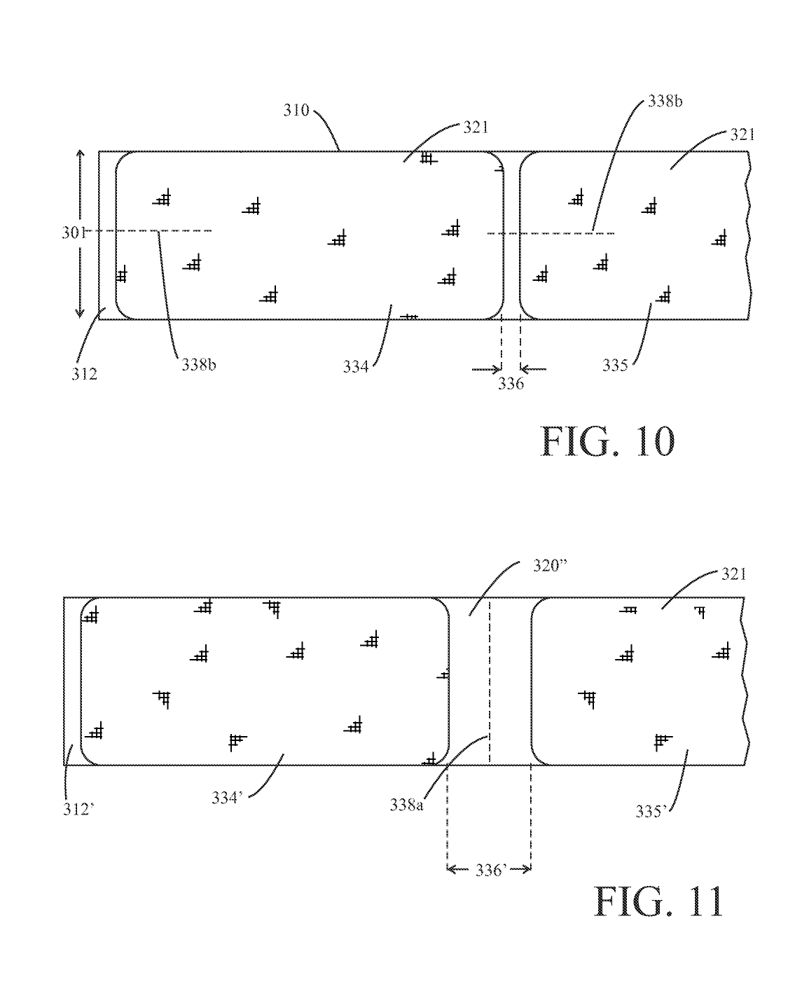

For use, a roll 300 of roll material 310 is disposed within the interior of the container 200. The roll material 310 has a lateral width 301 (shown in FIG. 10) that suitably dimensioned to permit dispensing of the roll material 310 from the container 200 through the first slit 212, and insertion of a lead end 312 of the roll material 310 into the container 200 through the second slit 213 for retention. Generally the lateral width of the roll material at the lead end is less than the height slit. For example, in a container 200 having first and second slit heights of 53 millimeters, the lateral width of the roll material 310 should be no more than about 52 millimeters.

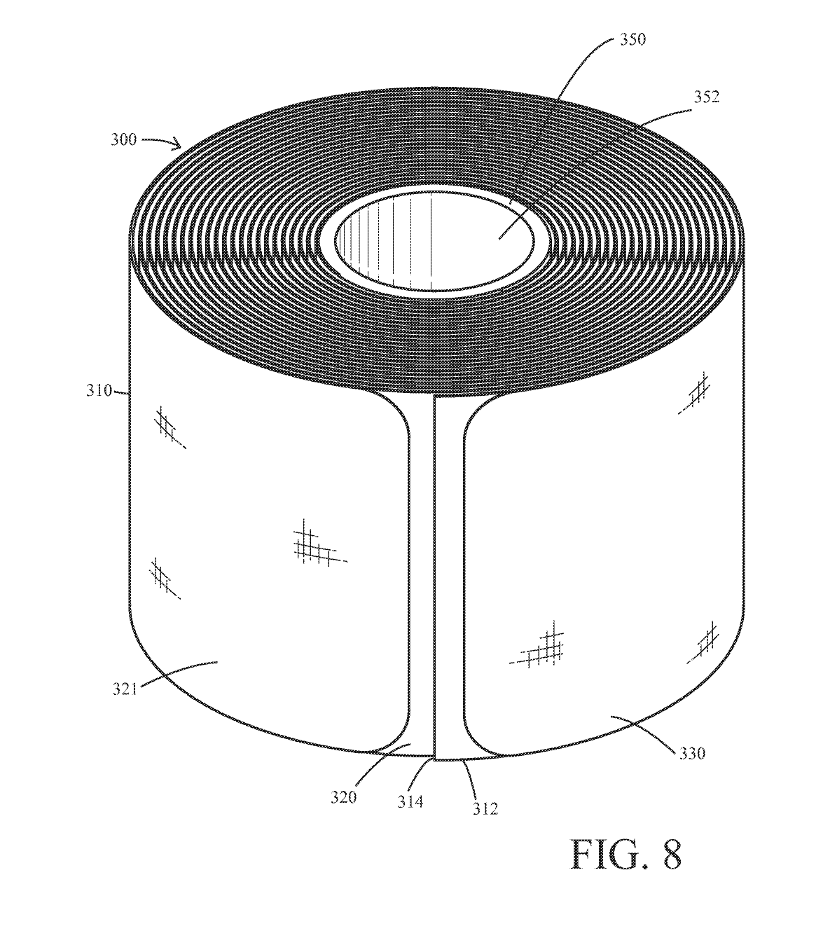

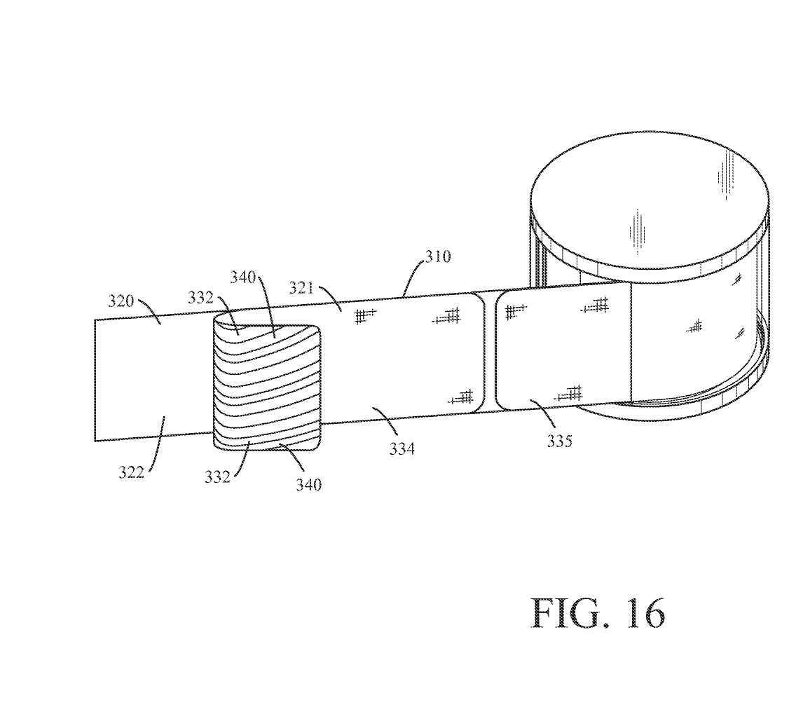

The dispenser may be used with any suitable roll material, including monolithic and compound roll materials. For example and with reference to FIGS. 8-11, the compared roll material 310 may comprise a backing material 320 and a working material 321 disposed on the backing material 320. The roll material 310 also includes an adhesive 340 disposed between the backing material 320 and the working material 321, as discussed further below. The adhesive 340 may include a contact adhesive to maintain contact between the working and backing materials, and may comprise, for example, a pressure-sensitive adhesive suitable for applying to skin, as is common with elastic therapeutic tape. With reference to FIG. 16, during manufacture of the roll material 310, the adhesive 340 may be applied to a support surface 322 of the backing material 320, or may be applied to a contact surface 332 of the working material 321, or may be applied to both the backing material 320 and the working material 321.

The backing material 320 may be any material that is suitable for supporting the working material 321 and otherwise functioning for the purposes described herein. Exemplary backing materials include paper, plastic film, cloth, fiber, fabric, metal foil, rubber, or any combination thereof. The backing material 320 may also include a release agent or a release liner to facilitate removal of the working material 321 therefrom. Preferably, the backing material 320 is a continuous strip of backing material that is, for example, between two and three meters in length.

The working material 321 may be a textile (e.g., wool, silk, cotton, nylon, polyester, or acrylic), synthetic fiber (e.g., nylon, spandex, polyester, acrylic, or polyolefin), latex, neoprene, rubber, silicone, or vinyl, or any combination thereof. Preferably, the working material 321 is elastic in at least the longitudinal direction such that the working material 321 can be stretched up to 210% of its resting longitudinal length, in many cases from 150% to 210% of the resting longitudinal length. The working material 321 preferably is water resistant. In a preferred approach, the working material 321 is an elastic, water resistant material comprised of 97.4% cotton and 2.6% spandex. In this way, the working material 321 is suitable for use as an elastic therapeutic tape. As depicted in FIG. 16, when configured as elastic therapeutic tape, the pressure-sensitive adhesive 340 is disposed in a pattern on the contact surface 332 of the strip 334 of working material 321, as is generally known in the art, for purposes of permitting airflow and accommodating muscle motion when applied.

The working material 321 may be provided as a plurality of discrete segments or strips (shown as 334, 335 in FIG. 16) disposed on the continuous strip of backing material 320. The individual strips may be comprised of a combination of working material 321 and adhesive 340 (for example, 76% working material 321 and 24% adhesive 340). The strips may have any suitable dimensions, for example, a longitudinal extension of between 150 millimeters and 300 millimeters preferably approximately 250 millimeters in length. The strips are capable of being individually removed from the backing material. Such strips may be generated using a die-cut process employing a roller having a cutting edge that extends from the roller to a height sufficient to cut the working material into discrete segments but not sufficient to cut into the backing material.

Preferably, as shown in FIG. 10, the ends of the strips 334 are separated from adjacent ends by intervening spaces or gaps (one shown as 336 in FIG. 10). The spaces 336 in one embodiment are between 3 millimeters and 6 millimeters in length, preferably is about 5 millimeters. Such spaces provide several advantages. For example, each space provides the user with a clear “tear point”; that is, the user can quickly identify where the backing material should be ripped to separate a desired portion from the roll of roll material. Furthermore, the spaces allow the user to manipulate one end of a strip 334 without interfering with another end of the next strip 335 of working material. In another embodiment, shown in FIG. 11, when container 200 and slits 212, 213 are dimensioned as described above, the spaces 336 may have a length between 45 and 55 millimeters, the reason for which is discussed in more detail below. In yet another alternative embodiment, no spaces are included between the strips. In this approach, each end of a discrete segment of working material abuts an adjacent end of a discrete segment of working material.

Referring now to FIG. 8, the roll of roll material 300 may be disposed on a bobbin 350. The bobbin 350 provides support against compressive forces to the roll of roll material 300 and also interacts with the protrusion 272 to retain the roll of roll material 300 radially when the container 200 is closed, in particular as the roll material 310 is consumed. In the illustrated embodiment, the protrusion 272 is sized to extent into a hollow 352 of the bobbin 350 to inhibit radial movement of the roll 300 within the container 200. In some cases the protrusion 272 may serve as a bearing for journaled rotation of the bobbin 350, particularly if the container 200 includes a complementary opposing protrusion. The axial retaining member 274 engages the bobbin 350 and/or the roll material 310 and inhibits the roll of roll material 300 from translating axially, thereby inhibiting undesired “rattle” of the roll of roll material within the container and maintaining the roll and slits in axial alignment.

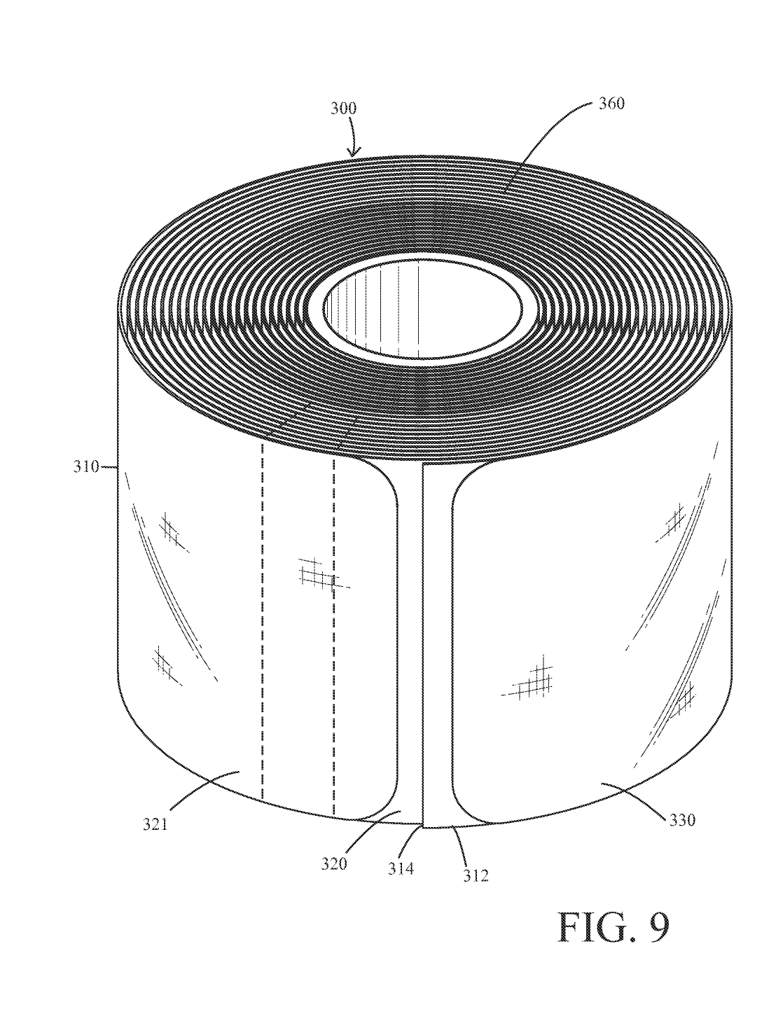

Referring now to FIG. 9, the roll of roll material 300 may be enclosed with a film or wrap 360, such as translucent shrink wrap for shipment. Alternatively, or in conjunction with the film or wrap, the roll of roll material 300 may be held in place by an adhesive patch (not shown).

Referring again to FIGS. 10 and 11, the backing material 320, the working material 321, or both, may include perforations 338a, 338b. Perforations 338a in the backing material 320 (shown in FIG. 11) facilitate removal of a portion of the roll material 310 from the roll of roll material 300. Such perforations 338a in the backing material 320 may be disposed immediately adjacent the ends of the strips, or may be disposed in the spaces 336′ between adjacent ends of the strips 334′, 335′. Perforations 338a in the backing material 320′ may also be disposed underneath the strips; for example, at 50 millimeters from the ends of the strips. This configuration allows the user to the remove only a portion of the backing material 320′, thereby exposing only a portion of the adhesive on the strip. In this way, the user can apply only the exposed portion of the strips without exposing the adhesive of the remainder of the strip. The working material 321 additionally or alternatively may include perforations 338b as shown in FIG. 10. Such perforations 338b allow the user to manipulate the shape of the strips prior to application.

In preparing the dispenser 100, the roll of roll material 300 is placed into an opening 212 of the container 200 and the cover 240 or 241 is installed over the opening 212. The container, cover, and roll of roll material 300 may be purchased in advance, or the invention alternatively may encompass preparing one or all of these items, for instance, by forming the container and for the cover from a resinous base material. When the container includes a roll of roll material disposed on a bobbin, the method of preparing the dispenser may comprise positioning the bobbin relative to the protrusion to inhibit axial movement of the roll.

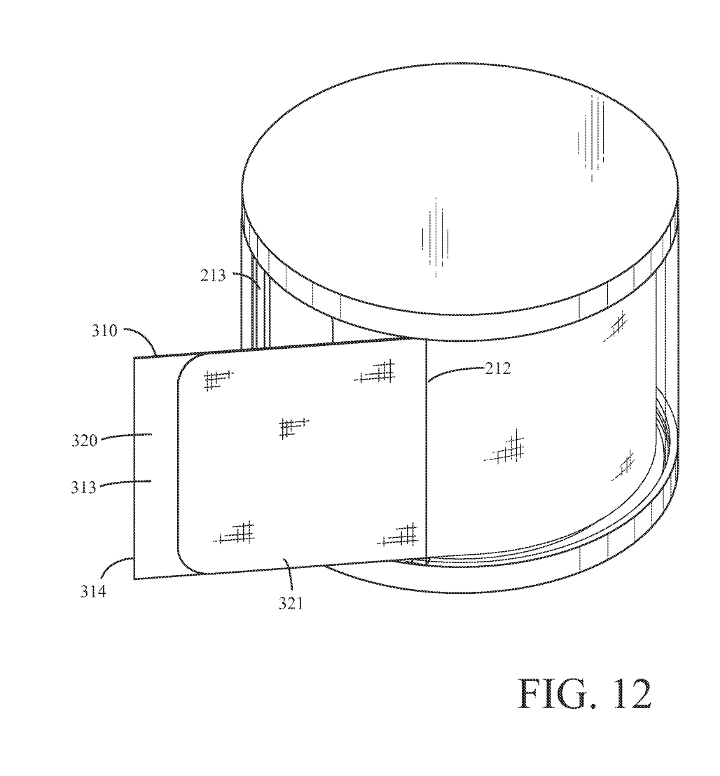

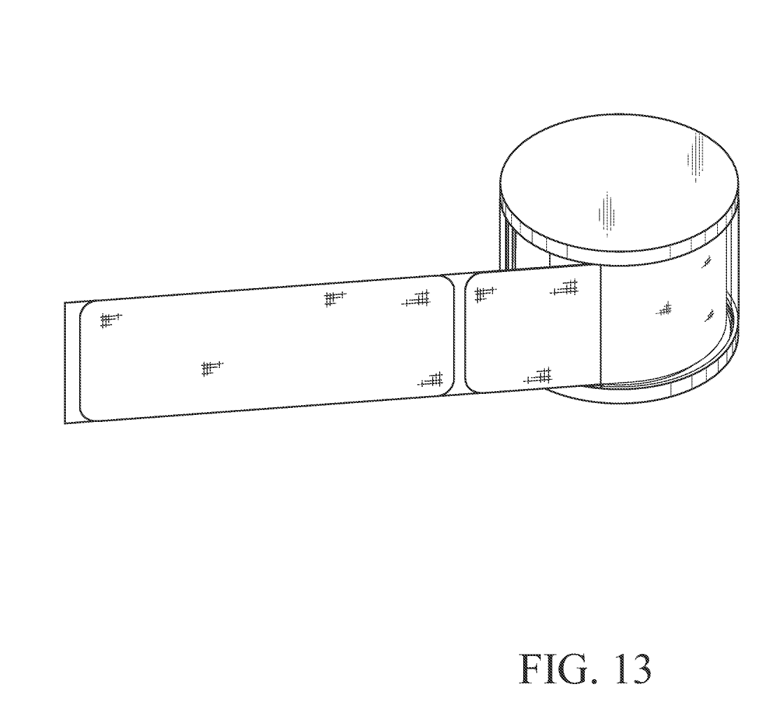

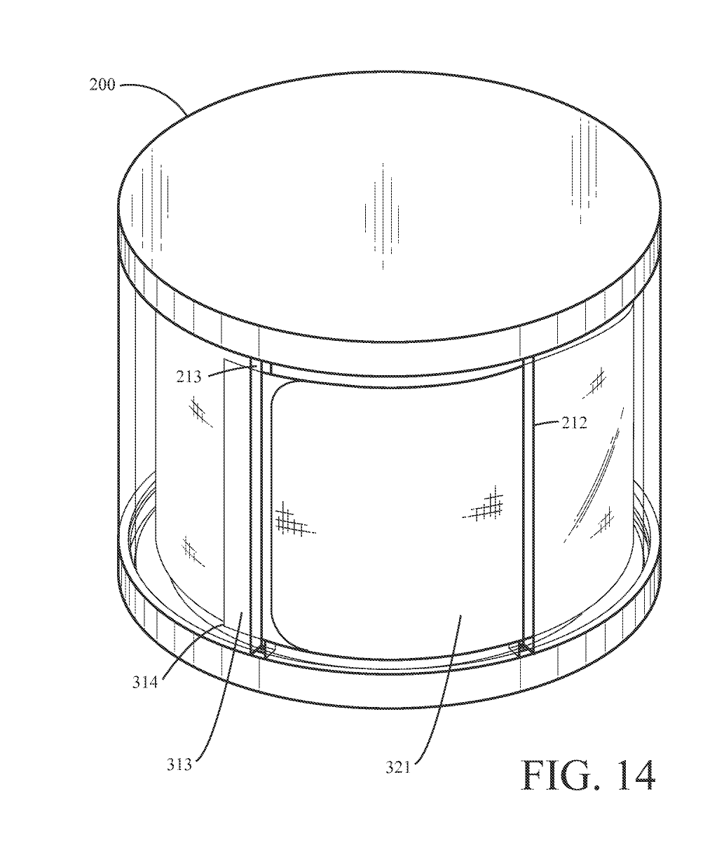

In use, as illustrated in FIGS. 12-14, if necessary the protective wrap is removed from the roll and the roll reinserted into the container. When a user desires to obtain roll material 310, a lead end 314 of the roll material 310 is dispensed through the first slit 212, and a portion of the dispensed roll material 310 is removed. This action contemplates either or both removing working material 321 from backing material 320 leaving backing material 320 on to the roll 300, or removing (e.g., by tearing) a strip of material that comprises backing material 320 and working material 321. In any case, this action leaves a new lead portion 313 of the roll material 310 (shown in FIG. 12) extending from the first slit 212, the lead portion 313 having a lead end 314. Thereafter, the lead end 314 is manually tucked into the second slit 213 to thereby retain the lead end 313, as shown in FIG. 14. The roll material therefore remains secure and is prevented from unraveling.

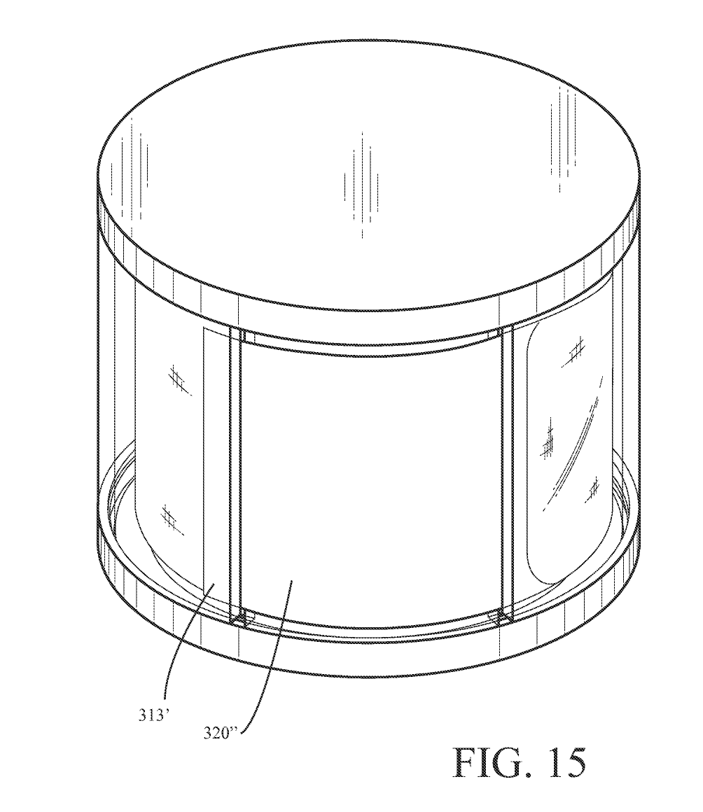

As seen, after the initial threading of the roll material 310 through the first slit 212, a portion of working material 321 remains exposed at the exterior of the container In an alternative approach, utilizing roll material 310 having a longer space 336′ as described above with respect to FIG. 11, only backing material 320′ is exposed when the lead end portion 313′ is tucked into the second slit, as seen in FIG. 15. This result alternatively may be achieved by reducing the spacing between the first and second slits.

The method may further include applying the roll material 310 or working material 321 to a body surface, e.g., by applying elastic therapeutic tape to the skin. The roll material dispenser 100 and method are particularly suitable for use with elastic therapeutic tape, although the invention is not limited thereto.

It is thus seen that a roll material dispenser and method of dispensing a roll material are provided, as is a matter for preparing a continuation of roll material.

Uses of singular terms such as “a,” “an,” are intended to cover both the singular and the plural, unless otherwise indicated herein or clearly contradicted by context. The terms “comprising,” “having,” “including,” and “containing” are to be construed as open-ended terms. Any description of certain approaches or embodiments as “preferred” approaches or embodiments, and other recitation of approaches, embodiments, features, or ranges as being preferred, or suggestion that such are preferred, is not deemed to be limiting. The invention is deemed to encompass embodiments that are presently deemed to be less preferred and that may be described herein as such. All methods described herein can be performed in any suitable order unless otherwise indicated herein or otherwise clearly contradicted by context. The use of any and all examples, or exemplary language (e.g., “such as”) provided herein, is intended to illuminate the invention and does not pose a limitation on the scope of the invention. Any statement herein as to the nature or benefits of the invention or of the preferred embodiments is not intended to be limiting. This invention includes all modifications and equivalents of the subject matter recited herein as permitted by applicable law. Moreover, any combination of the above-described elements in all possible variations thereof is encompassed by the invention unless otherwise indicated herein or otherwise clearly contradicted by context. The description herein of any reference or patent, even if identified as “prior,” is not intended to constitute a concession that such reference or patent is available as prior art against the present invention. No unclaimed language should be deemed to limit the invention in scope. Any statements or suggestions herein that certain features constitute a component of the claimed invention are not intended to be limiting unless reflected in the appended claims. Neither the marking of the patent number on any product nor the identification of the patent number in connection with any service should be deemed a representation that all embodiments described herein are incorporated into such product or service.

What is claimed is:

1. A roll material dispenser comprising:
a container comprising:
a generally cylindrical body defining a wall surface and a substantially hollow interior;
a cover connected to the cylindrical body; and
a first slit disposed in the wall surface and having a first slit height; and
a second slit disposed in the wall surface and having a second slit height, the second slit radially spaced from the first slit;
a roll of roll material disposed within the interior of the container, the roll material having a lateral width, the lateral width of the roll material and the first and second slit heights being suitably dimensioned to permit dispensing of the roll material from the container through the first slit and to permit a lead portion of the roll material to be tucked into the second slit to thereby retain the lead portion.

2. The roll material dispenser of claim 1, the cover being detachable from and reconnectable to the cylindrical body.

3. The roll material dispenser of claim 2, the cover being is threadably connectable to the cylindrical body.

4. The roll material dispenser of claim 1, the cover comprising a first cover, the container comprising a second cover opposing the first cover.

5. The roll material dispenser of claim 1, wherein the first and second slits are generally axially oriented.

6. The roll material dispenser of claim 5, wherein the first and second slits are substantially parallel.

7. The roll material dispenser of claim 1, the roll material comprising a backing material and a working material disposed on the backing material, the working material including a pressure-sensitive adhesive.

8. The roll material dispenser of claim 7, the working material comprising elastic therapeutic tape.

9. The roll material dispenser of claim 7, the roll material comprising a plurality of discrete segments of working material on a continuous roll of backing material.

10. The roll material dispenser of claim 9, wherein adjacent ends of the plurality of discrete segments are separated by an intervening gap.

11. The roll material dispenser of claim 1, the container being provided with a protrusion, the protrusion extending into the interior of the cylindrical body at least when the container is closed, the protrusion being sized and positioned to inhibit radial motion of the roll of roll material.

12. The roll material dispenser of claim 11, the protrusion being disposed on the cover, the cover comprising a base surface a and generally orthogonal lip surface that extends away from the base surface, and the protrusion extending away from the base surface to a greater distance away from the base surface than the lip surface.

13. The roll material dispenser of claim 12, the roll material being rolled on a bobbin, the bobbin having a hollow, the protrusion extending into the hollow of the bobbin at least when the container is closed.

14. The roll material dispenser of claim 13, the container including an axial retaining member sized and positioned to inhibit axial motion of the roll of roll material at least when the container is closed.

15. A method comprising:
providing a container that comprises a generally cylindrical body defining a wall surface, a substantially hollow interior, and an opening, the container further comprising first and second slits disposed in the wall surface and radially spaced from one another, the first slit having a first slit height, and the second slit having a second slit height;
providing a roll of roll material having a lateral width, the lateral width of the roll material and the first and second slit heights being suitably dimensioned to permit dispensing of roll material from the container through the first slit and to permit a lead portion of the roll material to be tucked into the second slit to thereby retain the lead portion;
inserting the roll of roll material into the container, and
covering the opening with a cover.

16. The method of claim 15, further comprising forming the container from a resinous base material.

17. The method of claim 15, the roll material comprising a backing material and a working material, the working material comprising elastic therapeutic tape.

18. The method of claim 15, the container being provided with a protrusion, the method including positioning the protrusion to extend into the interior of the cylindrical body, the protrusion being sized and positioned to inhibit radial motion of the roll of roll material.

19. The method of claim 15, the protrusion being disposed on the cover, the cover comprising a base surface a and generally orthogonal lip surface that extends away from the base surface, and the protrusion extending away from the base surface to a greater distance away from the base surface than the lip surface.

20. The method of claim 15, the roll material being rolled on a bobbin, the bobbin having a hollow, the method including positioning the protrusion to extend into the hollow.

21. A method comprising:
providing a roll material dispenser that comprises a container and a roll of roll material contained therein, the container comprising a generally cylindrical body defining a wall surface, and first and second slits disposed in the wall surface, the first slit having a first slit height, and the second slit having a second slit height, the roll material having a lateral width, the lateral width of the roll material and the first and second slit heights being suitably dimensioned to permit dispensing of the roll material from the container through the first slit and to permit a lead portion of the roll material to be tucked into the second slit to thereby retain the lead portion;
dispensing roll material through the first slit;
removing a portion of the dispensed roll material to leave a lead portion of the roll material extending from the first slit, the lead portion having a lead end;
tucking the lead end into the second slit to thereby retain the lead portion.

22. The method of claim 21, wherein the lead portion comprises a backing material and a working material disposed on the backing material, the working material comprising elastic therapeutic tape.

23. The method of claim 22, further comprising applying the elastic therapeutic tape to a body surface.
